(19) United States
(12) Patent Application Publication
(10) Pub. No.: US 2009/0247901 A1
(43) Pub. Date: Oct. 1, 2009

(54) LATCHING SIDE REMOVAL SPACER

(76) Inventor: Brian Zimmer, Indianapolis, IN (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 12/054,901

(22) Filed: Mar. 25, 2008

(51) Int. Cl. A61B 10/02

(52) U.S. Cl. 600/567

(57) ABSTRACT

A medical system includes a first medical device having a first engaging portion and a first cannula extending therefrom, a second medical device having a second engaging portion and a second cannula extending therefrom, an introducer having an introducer cannula and an axial distance adjustment system for providing an adjustable length between the introducer cannula distal end and at least a portion of one of the first engaging portion and the second engaging portion. The adjustment system includes at least one spacer having a side opening for permitting the at least one spacer to be interposed between the introducer and at least one of the first medical device and the second medical device. At least one of the first cannula and the second cannula may be selectively interposed within the side opening when at least a portion of the at least one of the first cannula and the second cannula is at least partially interposed within the introducer cannula.

### TECHNICAL FIELD

The present disclosure relates to the field of medical devices and more particularly to a medical system that permits introduction of, among other things, minimally invasive surgical instruments and other medical treatments into a patient's body.

### BACKGROUND

Medical procedures have advanced to stages where less invasive or minimally invasive surgeries, diagnostic procedures and exploratory procedures have become desired and demanded by patients, physicians, and various medical industry administrators. To meet these demands, improved medical devices and instrumentation have been developed, such as cannula or micro-cannula, medical introducers, vacuum assisted biopsy apparatus, and other endoscopic related devices.

In the field of tissue biopsy, minimally invasive biopsy devices have been developed that require only a single insertion point into a patient's body to remove one or more tissue samples. One such biopsy device incorporates a “tube-within-a-tube” design that includes an outer piercing needle having a sharpened distal end and a lateral opening that defines a tissue receiving port. An inner cutting member is slidingly received within the outer piercing needle, which serves to excise tissue that has prolapsed into the tissue receiving port. A vacuum is used to draw the excised tissue into the tissue receiving port and aspirates the excised tissue from the biopsy site once severed.

Exemplary “tube-within-a-tube” biopsy devices are disclosed in U.S. Pat. Nos. 6,638,235 and 6,744,824, which are owned by the assignee of the present invention. Among other features, the exemplary biopsy devices can be used in conjunction with Magnetic Resonance Imaging (MRI). This compatibility is due to the fact that many of the components of the biopsy devices are made of materials that do not interfere with operation of MRI apparatus or are otherwise compatible therewith. It is desirable to perform biopsies in conjunction with MRI because it is a non-invasive visualization modality capable of defining the margins of a tumor.

Some biopsy devices may incorporate an introducer having an introducer cannula that may be placed over the biopsy needle extending from about the biopsy location to a location outside the patient. This introducer may remain in place after a biopsy is taken to permit the biopsy needle to be removed and a marker deployment device to be inserted within the introducer cannula in order to permit a marker to be positioned within the biopsy site. However, with differing sizes of outer cannula for biopsy needles and marker deployment devices, undesirable amounts of leakage between the outer cannula and the biopsy needle and/or marker deployment device may exist.

Additionally, biopsy needles and introducers are available in differing lengths, which demands that marker deployment devices be capable of sliding within the introducer a predetermined length for proper marker deployment. While a removable annular spacer positioned between the introducer hub and the marker deployment device may permit the marker deployment device to be inserted to a predetermined depth, interposing the marker deployment device within an annular spacer may increase the risk of contamination. Additionally, a spacer interposed between the introducer hub and the marker deployment device may not secure the introducer hub to the marker deployment device, thereby requiring a user to simultaneously deploy a marker while ensuring that the marker deployment device is properly positioned axially with respect to the desired marker deployment location.

While the exemplary MRI compatible biopsy devices have proven effective in operation, in some procedures it may be desirable to temporarily latch a biopsy device or marker deployment device to an introducer. A favorable introducer may also reduce leakage through the introducer cannula and provide for adjustability for the insertion depth of the marker deployment device.

### BRIEF DESCRIPTION OF THE DRAWINGS

Referring now to the drawings, illustrative embodiments are shown in detail. Although the drawings represent some embodiments, the drawings are not necessarily to scale and certain features may be exaggerated, removed, or partially sectioned to better illustrate and explain the present invention. Further, the embodiments set forth herein are not intended to be exhaustive or otherwise limit or restrict the claims to the precise forms and configurations shown in the drawings and disclosed in the following detailed description.

FIG. 1 is a partially sectioned side view of a medical system according to an embodiment, with section graphics omitted for clarity.

FIG. 2 is a partially sectioned side view of a portion of the medical system of FIG. 1, with section graphics omitted for clarity.

FIG. 3 is a side view of a portion of a medical system according to another embodiment.

FIG. 4 is a partially sectioned side view of the medical system of FIG. 3.

FIG. 5 is a partially sectioned side view of the medical system of FIG. 4.

FIG. 6 is a partially sectioned side view of a medical system according to a further embodiment.

FIG. 7 is a perspective view of a portion of the medical system of FIG. 6.

FIG. 8 is a side view of a portion of the medical system of FIG. 6.

FIG. 9 is a partially sectioned side view of a medical system of FIG. 6, illustrating additional components.

FIG. 10 is a perspective view of a portion of a medical system according to an embodiment.

FIG. 11 is a perspective view of a portion of the medical system of FIG. 10.

FIG. 12 is a perspective view of a portion of the medical system of FIG. 10.

FIG. 13 is a partially sectioned side view of the device of FIG. 11.

FIG. 14 is a partially sectioned side view of a portion of the device of FIG. 11.

FIG. 15 is a perspective view of a portion of the medical system of FIG. 10.

FIG. 16 is a top view of the device of FIG. 15.

FIG. 17 is a side view of the device of FIG. 15.

FIG. 18 is a side view of the device of FIG. 15, rotated 90° from the view of FIG. 17.

### DETAILED DESCRIPTION

Referring, now to the drawings, the preferred illustrative embodiments of the present invention are shown in detail. Although the drawings represent some preferred embodiments of the present invention, the drawings are not necessarily to scale and certain features may be exaggerated to better illustrate and explain the present invention. Further, the embodiments set forth herein are not intended to be exhaustive or otherwise limit or restrict the invention to the precise forms and configurations shown in the drawings and disclosed in the following detailed description.

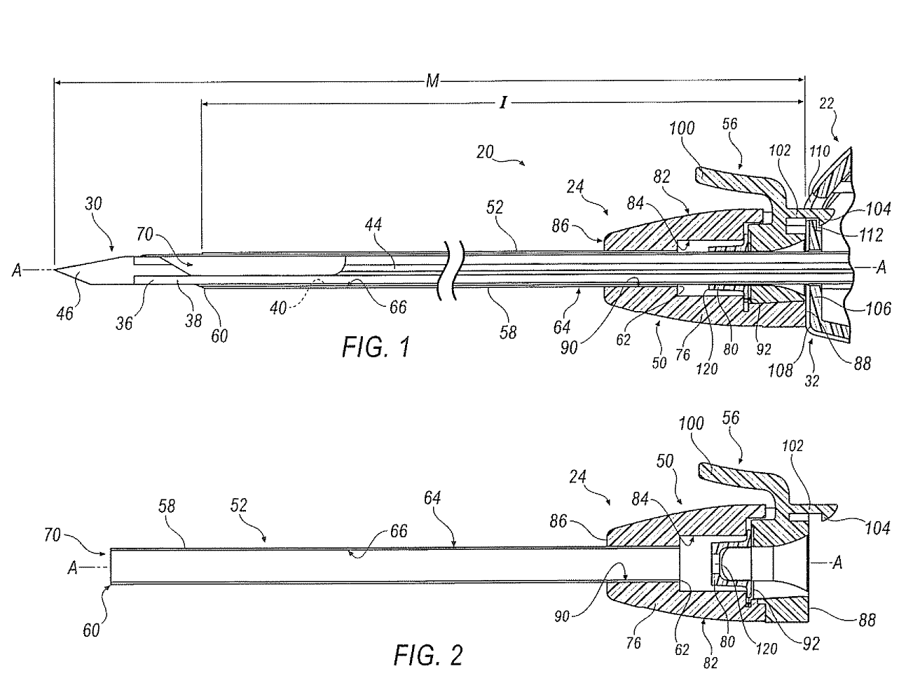

FIG. 1 illustrates a medical system 20. The medical system 20 includes a medical device, or biopsy device 22 (illustrated partially) and an introducer 24 generally defining an axis A-A. The biopsy device 22 includes a cutting element 30 sized for introduction into a patient's body and extends from a hand piece 32. The cutting element 30 includes an outer cannula 36 defined by a first outer lumen 38 and a first inner lumen 40, and an inner cannula 44 sized to fit concentrically within the first inner lumen 40. A motor or other motion generating device (not shown) may be provided with the hand piece 32 to rotate and/or translate inner cannula 44 within outer cannula 36. Biopsy apparatus similar to device 22 can be seen by way of example in U.S. Pat. Nos. 6,638,235 and 6,744,824, which are owned by the assignee of the present invention and are incorporated herein by reference in their entirety.

In the embodiment illustrated, the outer cannula 36 of the biopsy device 22 includes a tissue piercing tip 46, such as a trocar tip, to facilitate penetration of the system 20 into a patient's tissue. In addition to a trocar tip, it will be appreciated that the outer cannula 36 may include other devices for piercing the patient's tissue, including without limitation, devices that use a laser or radio frequencies (RF) to pierce the tissue.

As best seen in FIG. 2, the introducer 24 includes an introducer hub 50, an introducer cannula 52, and a latch portion 56. As will be described in detail, system 20 is particularly, but not necessarily, suited for use in biopsy procedures that identify the target biopsy site using Magnetic Resonance Imaging (MRI) or comparable medical imaging modality. The introducer 24 may be made of a MRI compatible, medical grade material, such as 316 stainless steel or Inconel™ 625.

The introducer cannula 52 includes a generally cylindrical body 58 having a distal end 60, a proximal end 62, an introducer outer lumen 64, and an introducer inner lumen 66. The distal end 60 defines a distal introducer opening 70. The hub 50 includes a generally annular hub portion 76, a hemostatic valve 80, and the latch portion 56. The annular hub portion 76 includes a hub outer surface 82, a hub inner surface 84, a hub distal end 86, and a hub proximal end 88. The hub inner surface 84 includes a generally cylindrical introducer cannula mating surface 90 and a generally cylindrical valve mating surface 92. The latch portion 56 includes a release button 100 and a latch 102 extending generally parallel to the axis A-A having a latch tab 104 extending generally perpendicular to and toward the axis A-A.

As best seen in FIG. 1, the biopsy device 22 includes a device distal end 106 defined by a distal surface 108, and a latch portion, or latch opening, 110. The latch opening 110 includes a latch tab interference portion 112.

As best seen in a comparison of FIGS, 1 and 2, the hemostatic valve 80 includes a body 120 that is a self-sealing membrane that will permit a medical device, such as the biopsy device 24 or a site marker deployment device, to pass therethrough while sealing around the medical device and will reseal with itself after the medical device is removed from the valve 80.

A medical device, such as the biopsy device 22 partially interposed within the introducer 24, may include a vacuum source (not shown). The vacuum source may aspirate the biopsy site where the biopsy device 22 removes a tissue sample.

The length of the outer cannula 36, from the distal surface 108 to the piercing tip 46 is identified by the reference character “M” in FIG. 1. The length of the introducer 24 from the distal end 60 to the hub proximal end 88 is identified by the reference character “I” in FIG. 1.

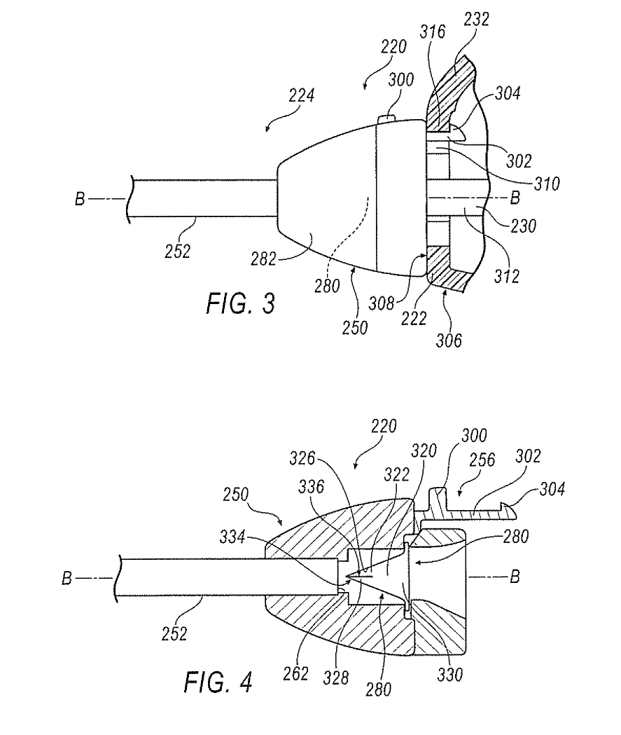

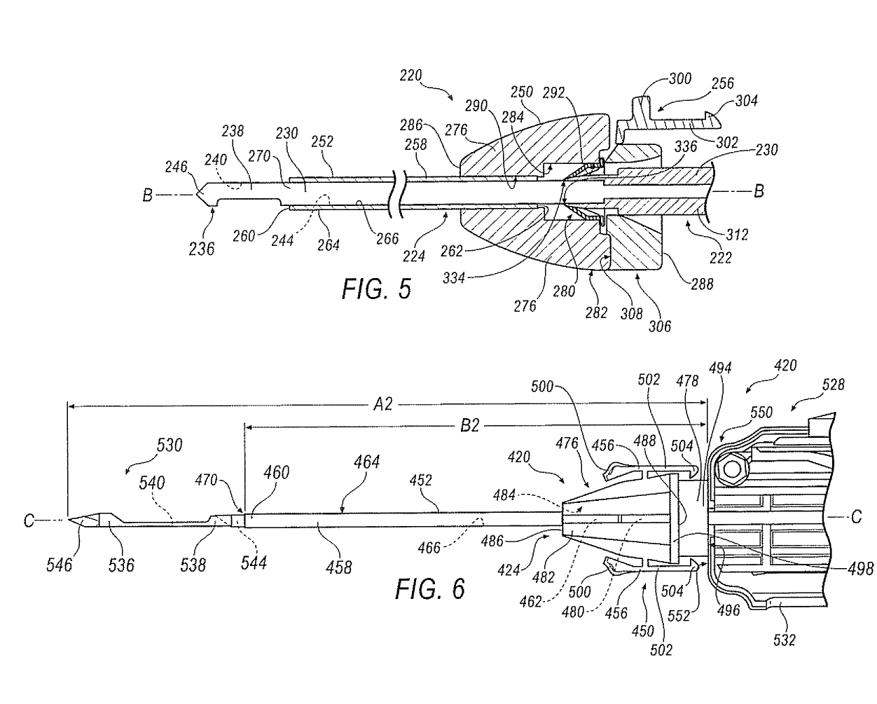

FIGS. 3-5 illustrate an alternative embodiment of the medical system 20 as a medical system 220. The medical system 220 includes a medical device, or biopsy device 222 (illustrated partially in FIGS. 3 and 5) and an introducer 224 generally defining an axis B-B. The biopsy device 222 includes a cutting element 230 that extends from a hand piece 232. The cutting element 230 includes an outer cannula 236 defined by a first outer lumen 238 and a first inner lumen 240, and an inner cannula 244 sized to fit concentrically within the first inner lumen 240. A motor or other motion generating device may be provided with the hand piece 232 to rotate and/or translate inner cannula 244 within outer cannula 236.

In the embodiment illustrated, the outer cannula 236 of the biopsy device 722 includes a tissue piercing tip 246, such as a trocar tip, to facilitate penetration of the system 220 into a patient's tissue. In addition to a trocar tip, it will be appreciated that the outer cannula 236 may include other devices for piercing the patient's tissue, including without limitation, devices that use a laser or radio frequencies (RF) to pierce the tissue.

As best seen in FIG. 4, the introducer 224 includes a hub 250, an introducer cannula 252, and a latch portion 256. As will be described in detail, system 220 is particularly, but not necessarily, suited for use in biopsy procedures that identify the target biopsy site using Magnetic Resonance Imaging (MRI) or comparable medical imaging modality.

As best seen in FIG. 5, the introducer cannula 252 includes a generally cylindrical body 258 having a distal end 260, a proximal end 262, an introducer outer lumen 264, and an introducer inner lumen 266. The distal end 260 defines a distal introducer opening 270. The hub 250 includes a generally annular hub portion 276, a hemostatic valve 280, and the latch portion 256. The annular hub portion 276 includes a hub outer surface 282, a hub inner surface 284, a hub distal end 286, and a hub proximal end 288. The hub inner surface 284 includes a generally cylindrical introducer cannula mating surface 290 and a generally cylindrical valve mating surface 292. The latch portion 256 includes a release button 300 and a latch 302 extending generally parallel to the axis B-B having a latch tab 304 extending generally perpendicular to the axis B-B.

As best seen in FIG. 3, the biopsy device 222 includes a device distal end 306 defined by a distal surface 308, a latch opening 310, and an outer cannula sheath 312. The latch opening 310 includes a latch tab interference portion 316.

As best seen in a comparison of FIGS. 4 and 5, the hemostatic valve 280 includes a body 320 having a slit 322 formed therein. The slit 322 generally segregates the body 320 into a first flap 326 and a second flap 328 interconnected at an outer periphery, or outer edge, 330 such that the slit 322 does not intersect the outer edge 330. The first flap 326 is defined by a first flap opening surface 334, and the second flap 328 is defined by a second flap opening surface 336. The first flap opening surface 334 and the second flap opening surface 336 are formed so as to flex inwardly until the first flap opening surface 334 and the second flap opening surface 336 bindingly contact (FIG. 4) and provide a seal for the introducer inner lumen 266. To provide this resilient flexing for a self-sealing effect, the valve 280 may be made of a silicone or other suitable material that will bias the first flap 326 and the second flap 328 toward a closed position.

The first flap opening surface 334 and the second flap opening surface 336 are in contact in the closed position of FIG. 4 and provide a seal for the introducer inner lumen 266 when the valve 280 does not have a medical device interposed therein. In FIG. 5, the first flap opening surface 334 and the second flap opening surface 336 contact the first outer lumen 238 so as to form a seal therebetween and restrict fluids from leaking therepast and through the introducer cannula 252. In the embodiment illustrated, the hemostatic valve 280 is not punctured with each use, but is a valve having a defined opening.

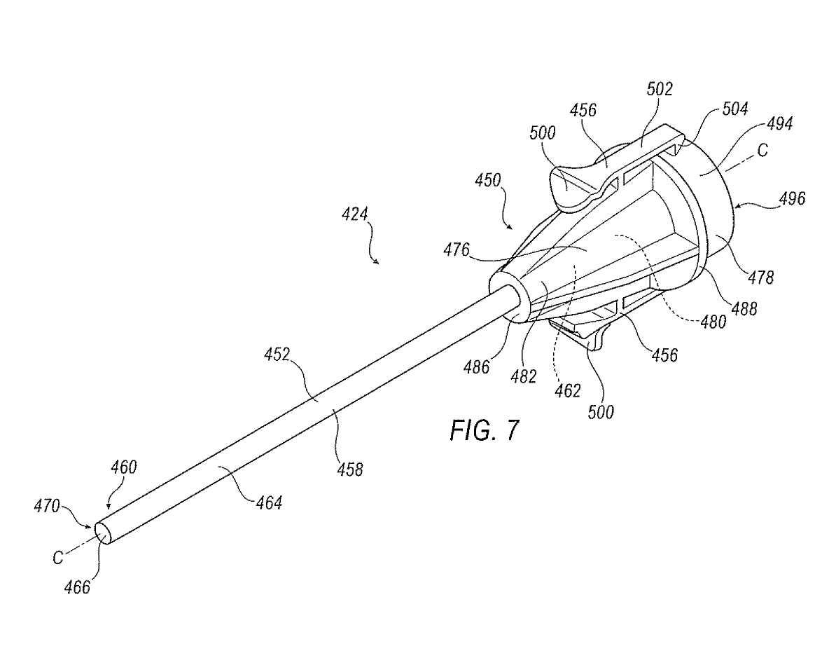

FIGS. 6-9 illustrate an alternative embodiment of the medical system 20 as a medical system 420. The medical system 420 includes a medical device, or site marker deploymnent device 422 (illustrated partially in FIG. 9) and an introducer 424 generally defining an axis C-C.

As best seen in the embodiment of FIG. 9, the site marker deployment device 422 includes a deployment handpiece 430, a deployment rod 432, and a deployment cannula 434 extending therefrom. The deployment cannula 434 includes a generally cylindrical body 436 having a distal deployment end 438 defined, at least in part, by a distal deployment opening 440, a proximal deployment end 442, a deployment inner lumen, or inner lumen, or inner surface, 444, and a deployment outer surface, 446. In the embodiment illustrated, the deployment inner lumen 444 and the deployment outer lumen 446 are generally cylindrical.

The deployment cannula 434 is illustrated in FIG. 9 with a site marker 448 (illustrated in phantom) interposed therein. The site marker 448 may be an MRI identifiable marker, such as a collagen plug, metal spring, or other medical treatment. The deployment rod 432 extends at least partially through the hand piece 430 and the deployment cannula 434 and is used to urge the site marker 448 through the distal deployment opening 440 when the deployment device 422 is desirably positioned, as discussed in greater detail below.

As best seen in FIGS. 6-9, the introducer 424 includes a hub 450, an introducer cannula 452, and a pair of latch portions 456. As will be described in detail, system 420 is particularly, but not necessarily, suited for use in biopsy procedures that identify the target biopsy site using Magnetic Resonance Imaging (MRI) or comparable medical imaging modality.

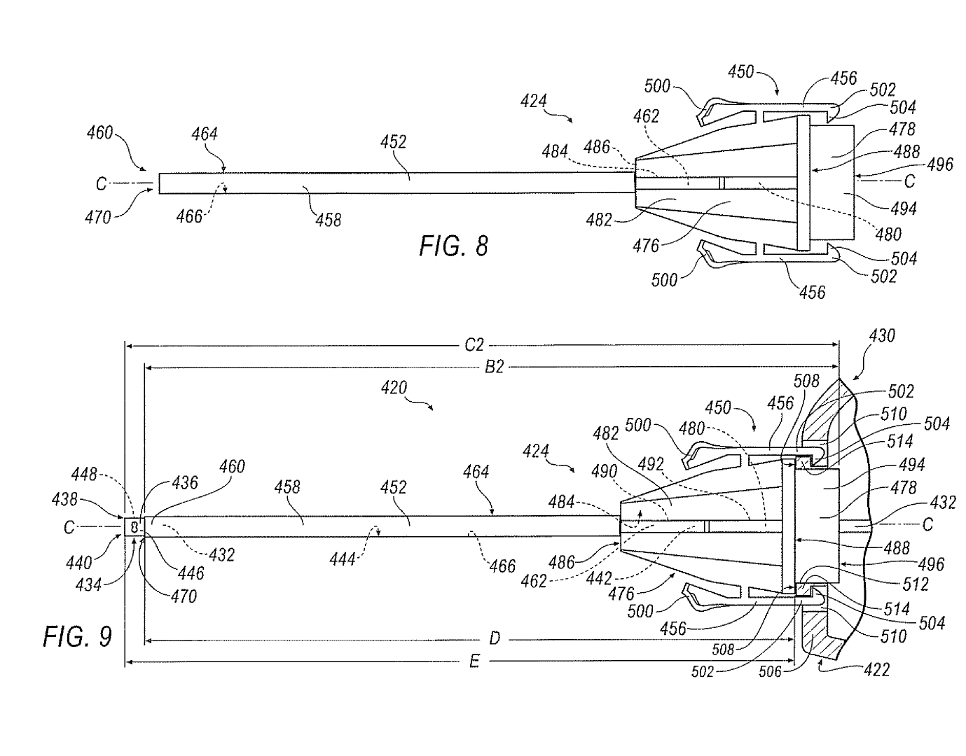

As best seen in FIG. 8, the introducer cannula 452 includes a generally cylindrical body 458 having a distal end 460, a proximal end 462, an introducer outer lumen 464, and an introducer inner lumen 466. The distal end 460 defines a distal introducer opening 470. The hub 450 includes a generally annular hub portion 476, a first portion, or collar, 478, a hemostatic valve 480, and the latch portions 456. The annular hub portion 476 includes a hub outer surface 482, a hub inner surface 484, a hub distal end 486, and a hub proximal end 488. The hub inner surface 484 includes a generally cylindrical introducer cannula mating surface 490 (FIG. 9) and a generally cylindrical valve mating surface 492 (FIG. 9). The collar 478 includes a generally cylindrical outer surface 494 and a generally annular collar end surface 496. The hub proximal end 488 includes a generally cylindrical hub flange 498. Each latch portion 456 includes a release button 500 and a latch 502 extending generally parallel to the axis C-C having a latch tab 504 extending generally perpendicular to the axis C-C.

As best seen in FIG. 9, the deployment handpiece 430 of the site marker deployment device 422 includes a deployment distal end 506 defined by a deployment distal surface 508. The deployment distal end 506 has a pair of latch openings 510 and a collar opening 512 formed therein. Each latch opening 510 includes a latch tab interference portion 514. The collar 478 is received within the collar opening 512. The hemostatic valve 480 nay be a valve 80 or a valve 280, as desired.

FIG. 6 illustrates the introducer 424 with a medical device, or a biopsy device 528 interposed therein. The biopsy device 528 includes a cutting element 530 sized for introduction into the patient's body. The cutting element 530 extends from a handpiece 532. The cutting element 530 includes an outer cannula 536 defined by a first outer lumen 538 and a first inner lumen 540, and an inner cannula 544 sized to fit concentrically within the first inner lumen 540. A motor or other motion generating device may be provided with the hand piece 532 to rotate and/or translate inner cannula 544 within outer cannula 536.

In the embodiment illustrated, the outer cannula 536 of the biopsy device 528 includes a tissue piercing tip 546, such as a trocar tip, to facilitate penetration of the system 520 into a patient's tissue. In addition to a trocar tip, it will be appreciated that the outer cannula 536 may include other devices for piercing the patient's tissue, including without limitation, devices that use a laser or radio frequencies (RF) to pierce the tissue.

The handpiece 532 includes a biopsy device distal end 550 having a biopsy device distal surface 552 for abutting the collar 478 to restrict the movement of the introducer 424 relative to the biopsy device 528. When the biopsy device 528 and the introducer 424 are coupled such as shown in FIG. 6, the length of the biopsy device 528, from the collar end surface 496 to the piercing tip 546 is identified by the reference character “A2” in FIG. 6.

The length of the introducer 424 from the distal end 460 to the collar end surface 496 is identified by the reference character “B2” in FIGS. 6 and 9. When the deployment device 422 and the introducer 424 are coupled such as shown in FIG. 9, the length of the deployment device 422, from the collar end surface 496 to the distal deployment opening 440 is identified by the reference character “C2” in FIG. 9. The length of the introducer 424 from the distal end 460 to the hub proximal end 488 is identified by the reference character “D” in FIG. 9. When the deployment device 422 and the introducer 424 are coupled such as shown in FIG. 9, the length of the deployment device 422, from the hub proximal end 488 to the distal deployment opening 440 is identified by the reference character “E” in FIG. 9.

In operation, a biopsy device, such as the biopsy device 528 is coupled with the introducer 424 such that the outer cannula 536 is interposed within the introducer cannula 452 with the piercing tip 546 extending from the distal introducer opening 470, as generally shown in FIG. 6. The biopsy device 528 is inserted into the introducer 424 until the collar end surface 496 contacts the biopsy device distal surface 552. In this biopsy configuration, the system 420 may be inserted into a patient's tissue to remove a tissue sample from a biopsy site. Also in this biopsy configuration, the valve 480 seals the introducer cannula such that fluids are restricted from flowing from the distal end 460 to the proximal end 462.

Next, the system 420 is inserted into a patient's tissue to a desired depth. This desired depth may be determined by viewing the system with a MRI during insertion. With the cutting element 530 positioned as desired, a tissue sample is drawn into the outer cannula 536 and separated from the surrounding tissue to form a biopsy site. A vacuum drawn through the outer cannula 536 may be applied to facilitate a complete separation and collection of the tissue sample.

Next, the biopsy device 528 is removed from the tissue as the introducer 424 is maintained in a relatively stable position relative to and within the tissue. As the piercing tip 546 passes the valve 480, the valve 480 seals with itself to restrict a loss of fluids from tile biopsy site. In the embodiment described, the valve 480 is a valve 280 where the first flap opening surface 334 and the second flap opening surface 336 flex inwardly until the first flap opening surface 334 and the second flap opening surface 336 bindingly contact (FIG. 4) and provide a seal for the introducer inner lumen 466.

Next, the deployment device 422, with a site marker 448 interposed therein, may be inserted into the introducer 424 (FIG. 9). The deployment device 422 is inserted into the introducer 424 until the deployment distal surface 508 contacts the hub proximal end 488. The deployment cannula 434 is sized to fit within the introducer cannula 452, but need not be snugly fit, since the valve 280 will reduce leakage therebetween.

The site marker 448 may then be deployed by urging the site marker out of the introducer 424 through the distal introducer opening 470. Deployment devices for deploying a site marker may be found in U.S. Pat. No. 7,044,957.

The deployment device 422 and the introducer 424 may be removed simultaneously by urging the deployment handpiece 430 away from the tissue generally in a direction parallel to the axis C-C since the deployment device 422 is latched to the introducer 424. Alternately, the deployment device 422 may be unlatched from the introducer 424 by urging the release buttons 500 inwardly toward the axis C-C to disengage the latch tabs 504 from the latch openings 510 and urge the deployment device 422 away from the introducer 424.

As illustrated and described herein the valve 280 (which may be commonly referred to as a duck bill valve) will permit medical devices to be inserted therethrough while restricting the flow of fluids therethrough. Either a biopsy device or a site marker deployment device, or both, could be latched to an introducer using a latch as described herein, as desired. The latches described herein permit a medical device to be positioned relative to an introducer hub in a desirable, confirmable position for performing a treatment, such as removing tissue or deploying a site marker or other treatment. An introducer hub, such as the introducer hub 50, 250, 450 may be positioned relative to the tissue by an indicator on the introducer outer lumen 464, or a support grid affixed to a MRI device. Additionally, the operation of the systems 20, 220 are similar to the system 420, with variations in whether the biopsy device or the deployment device (or both) are latched and unlatched from the introducer hub, as desired.

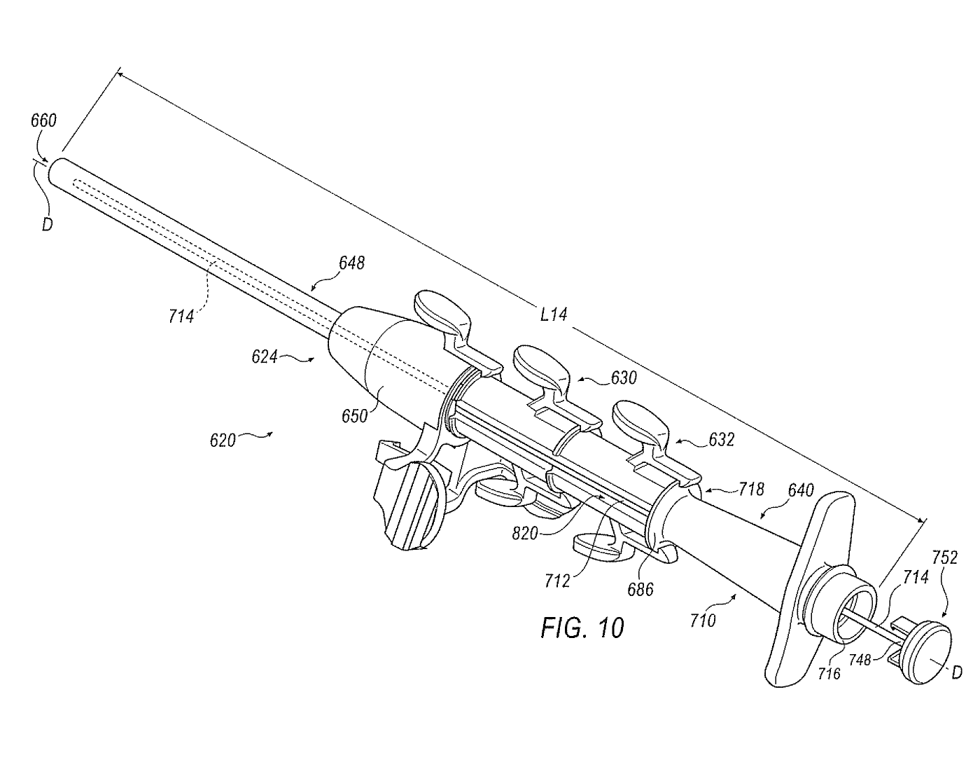

FIGS. 10-18 illustrate embodiments of the system 20 with adjusting mechanisms for accommodating cannula of differing lengths. That is, briefly, biopsy devices and marker deployment devices may be supplied with lengths of, for example, 10 centimeters (cm), 12 cm, or 14 cm. While introducers with lengths of 10, 12, and 14 cm may be supplied, a user may require a 12 cm introducer cannula for a biopsy device (such as the dimension B2 of the biopsy device 22 of FIG. 6) and a 14 cm introducer cannula for a site marker deployment device (such as the dimension B2 of the site marker deployment device of FIG. 9).

Since the introducer is generally not removed during the procedure of removing a biopsy device and deploying a marker for precision of marker positioning, the user may attempt to insert the deployment device partially, estimate when the deployment device is 2 cm from full insertion into an introducer (where full insertion is shown FIG. 9), and deploy the marker. This method may not provide the desired precision of marker positioning. Further, a user may stock multiple deployment devices having cannula of differing lengths to precisely deploy a marker depending upon the cannula length of the biopsy device employed.

FIG. 10 illustrates a portion of an embodiment of a medical system 620. The medical system 620 includes the biopsy device 22 (illustrated partially in FIG. 1), an introducer system 624, a first spacer 630, a second spacer 632, a marker deployment device 640, and an introducer 648.

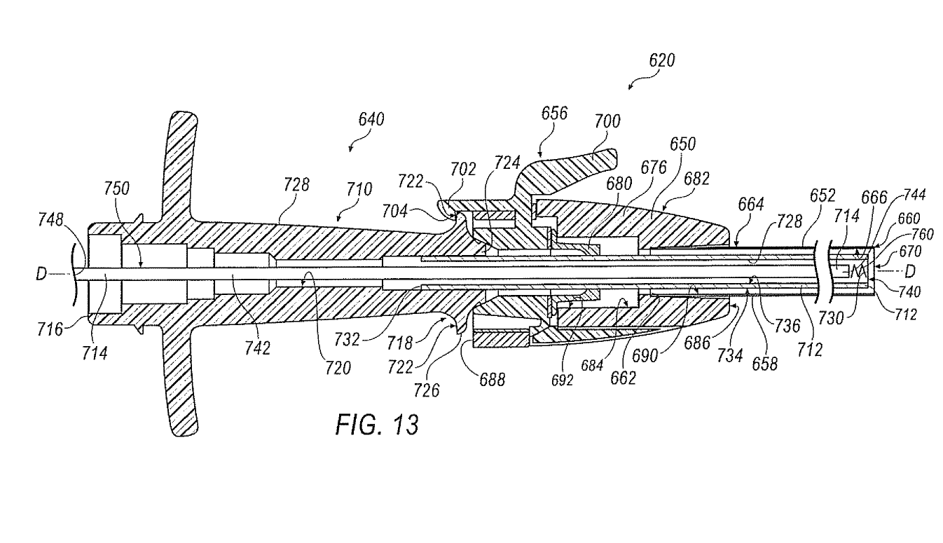

As best seen in FIG. 13, the introducer 648 includes an introducer hub 650, an introducer cannula 652, and a latch portion 656. As will be described in detail, system 620 is particularly, but not necessarily, suited for use in biopsy procedures that identify the target biopsy site using Magnetic Resonance Imaging (MRI) or comparable medical imaging modality. The introducer system 624 may be made of a MRI compatible, medical grade material, such as 316 stainless steel or Inconel™ 625.

The introducer cannula 652 includes a generally cylindrical body 658 having an introducer distal end 660, a proximal end 662, an introducer outer surface 664, and an introducer inner surface 666. The introducer distal end 660 defines a distal introducer opening 670. The hub 650 includes a generally annular hub portion 676, a hemostatic valve 680, and the latch portion 656. The annular hub portion 676 includes a hub outer surface 682, a hub inner surface 684, a hub distal end 686, and a hub proximal end 688. The hub inner surface 684 includes a generally cylindrical introducer cannula mating surface 690 and a generally cylindrical valve mating surface 692. The latch portion 656 includes a release button 700 and a latch portion 702 extending generally parallel to an axis D-D having a latch tab 704 extending generally perpendicular to and toward the axis D-D.

As best seen in FIG. 13, the hemostatic valve 680 includes a body that is a normally closed self-sealing membrane that will permit a medical device, such as the biopsy device 22 or a site marker deployment device, to pass therethrough while sealing around a portion of the medical device, such as the outer cannula 36. The hemostatic valve 680 will automatically reseal with itself after the medical device is removed from the hemostatic valve 680.

The marker deployment device 640 includes a marker deployment hub 710 a marker cannula 712 (FIG. 13), and a push rod 714. The marker deployment hub 710 includes a proximal end 716 a distal end 718 a marker deployment hub inner surface 720, a first latch surface 722, and a second latch surface 724. In the embodiment illustrated, the latch surface 722 is a generally annular, contoured collar 726 that extends from the distal end 718, although other suitable surfaces may be used.

The marker cannula 712, as best seen in FIG. 13, is coupled to the marker deployment hub 710 and includes a generally cylindrical body 728 having a deployment distal end 730, a proximal end 732, a deployment outer surface 734, and a deployment inner surface 736. The deployment distal end 730 defines a distal deployment opening 740. The push rod 714 includes a generally cylindrical elongated body 742 having a distal push rod end 744, a proximal push rod end 748, a generally cylindrical push rod outer surface 750, and a button 752 (FIG. 10) formed at the proximal push rod end 748. A marker 760 is illustrated interposed within the marker cannula 712 at the deployment distal end 730.

As also best illustrated in FIG. 13, as the marker deployment device 640 is coupled with the introducer 648, the marker cannula 712 is interposed axially generally along axis D-D through the introducer cannula 652 as the deployment distal end 730 extends to about the same axial position as the introducer distal end 660. When the deployment distal end 730 is in a desired axial position relative to the introducer distal end 660, the latch portion 656 of the introducer 648 will releasably latch to the latch surface 722 of the marker deployment device 640. The push rod 714 will translate within the marker cannula 712 such that the distal push rod end 744 will extend to the deployment distal end 730 and force the marker 760 out of the marker cannula 712, as discussed in greater detail below.

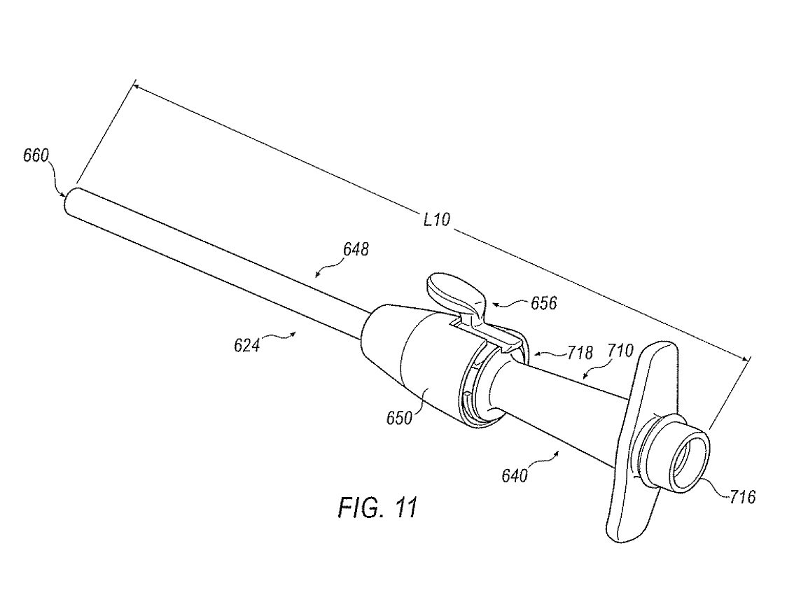

The length of the introducer system 624, as assembled in FIG. 10 with spacers 630, 632, from the hub distal end 686 to the introducer distal end 660 is identified by the reference character “L14” in FIG. 10. The length of the introducer system 624, as assembled in FIG. 11, from the introducer distal end 660 to the hub proximal end 716 is identified by the reference character “L10” in FIG. 11. As illustrated, the introducer system 624, in the configuration of FIG. 10 is longer than the configuration of FIG. 11. In the embodiment illustrated, each spacer 630, 632 will preferably lengthen the effective introducer span about 2 centimeters, L14 is about 14 cm and L10 is about 10 cm.

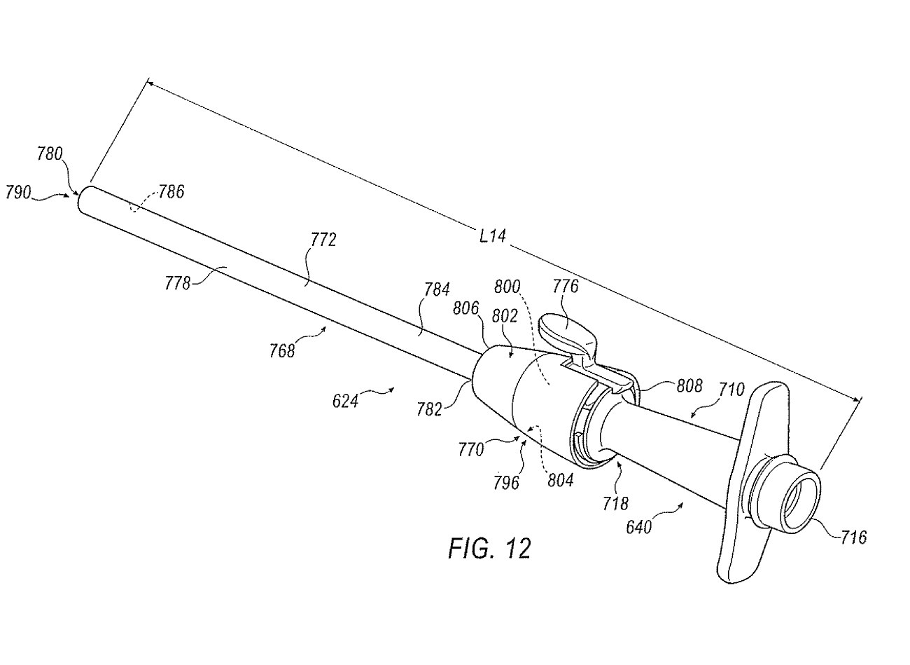

FIG. 12 illustrates another configuration of the introducer system 624. In FIG. 12, the introducer system 624 includes an introducer 768 having a hub 770, an introducer cannula 772, and a latch portion 776. The introducer cannula 772 includes a generally cylindrical body 778 having a distal end 780, a proximal end 782, an introducer outer surface 784, and an introducer inner surface 786. The distal end 780 defines a distal introducer opening 790. The hub 770 includes a generally annular hub portion 796, a hemostatic valve 800, and the latch portion 776. The annular hub portion 796 includes a hub outer surface 802, a hub inner surface 804, a hub distal end 806, and a hub proximal end 808.

As may be seen in a comparison of the embodiments illustrated in FIGS. 10 and 12, the introducer 648 is axially shorter than the introducer 768, As illustrated, the length of the introducer system of FIG. 10 (from the distal end 660 to the proximal end 716 is about the same length (identified by reference character L14) as the introducer system 624 of FIG. 12 from the proximal end 716 to the distal end 780. Accordingly, the spacers 630, 632 permit the introducer system 624 to selectively provide differing lengths, such as the lengths L10 and L14 of FIGS. 10 and 11, for the medical system 620, as desired. Further, additional lengths may be provided by using any number of spacers.

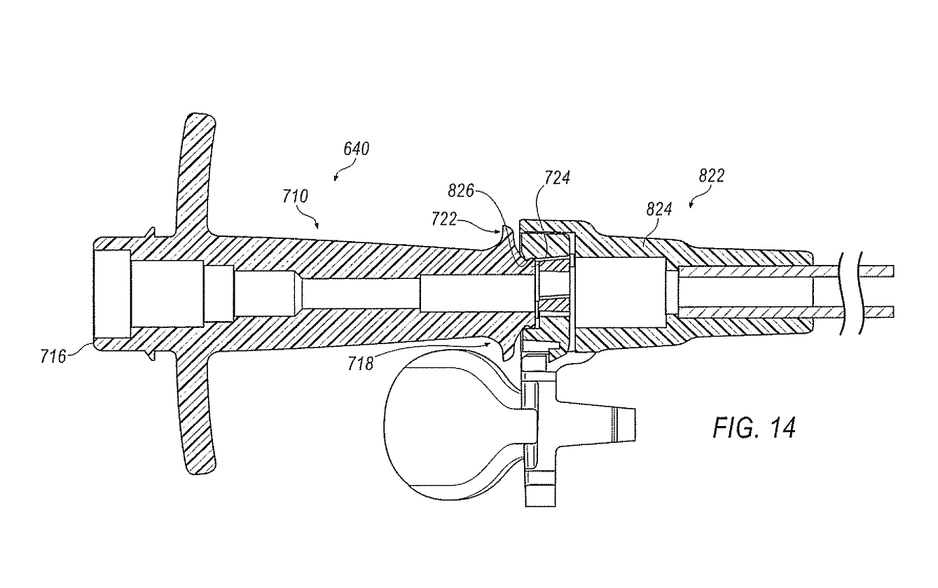

FIG. 14 illustrates the marker deployment device 640 releasably coupled to an introducer 822. The introducer 822 includes an introducer hub 824 having a generally annular contoured coupling surface 826. The surface 826 will releasably couple with the second latch surface 724 of the hub 710.

FIGS. 13 and 14 illustrate the marker deployment device 640 in differing configurations. While FIG. 13 is a partially sectioned view of FIG. 11, FIG. 14 illustrates the marker deployment device 640 releasably coupled with the introducer hub 824. As illustrated, the introducer hub 770 and the introducer hub 824 have differing geometries at their proximal ends, although each may releasably couple with the marker deployment device 640.

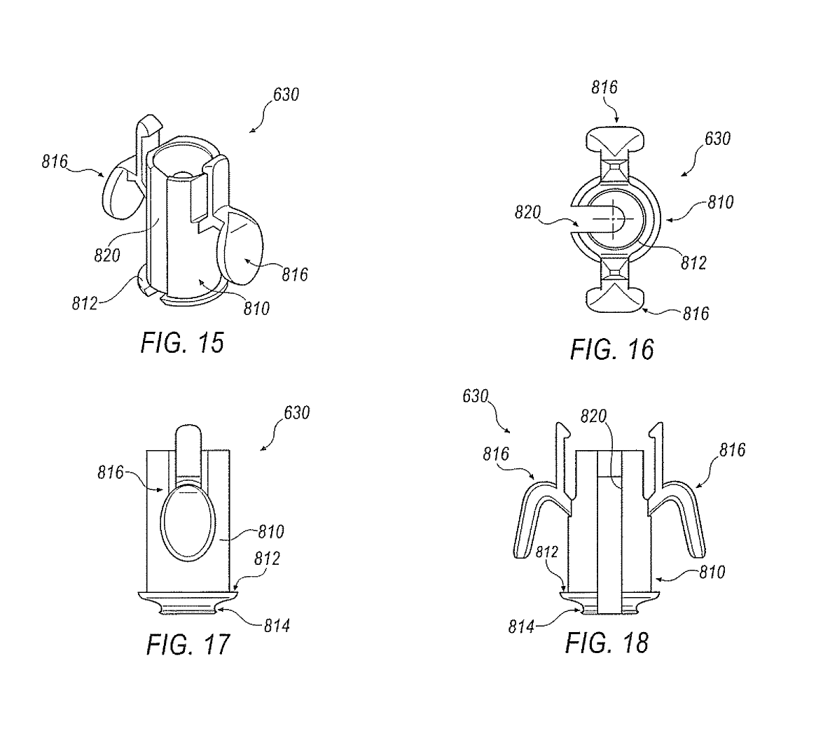

FIGS. 15-18 illustrate the spacer 630 in greater detail. In the embodiment illustrated, the spacer 630 is generally identical to the spacer 632 and includes a generally tubular body 810 having a first latch surface 812 and a second latch surface 814 formed thereon, and at least one latch portion 816 extending therefrom, and a slot 820 formed therein. As illustrated in FIGS. 15-18 and best seen in FIG. 10, the latch portions 816 are similar to the latch portion 776, and the latch surface 814 is similar to the latch surface 722, thereby permitting the spacers 630, 632 to releasably couple with both the hub 650 of the introducer 648 and the marker deployment hub 710.

As best illustrated in FIG. 10, the marker cannula 712 is seen interposed within the spacers 630, 632. In use, a user may insert the marker cannula 712 into the introducer 648 prior to coupling the spacers 630, 632 to either the hub 650 of the introducer 648 or the marker deployment hub 710. After inserting the marker cannula 712 into the introducer 648, the spacers 630, 632 are superposed onto the marker cannula 712 as the marker cannula 712 is interposed within the slots 820. The dimensions of the slot 820 (FIG. 16) are such that the marker cannula 712 (FIG. 13) may be selectively interposed therein (FIG. 10). Further, the marker cannula 712 may be dimensioned for an interference fit within the slot 820 to permit the marker cannula 712 to be “snapped” onto the slot 820 to couple the spacer 630 to the marker cannula 712 and prevent spacers 630 from undesirably falling from the marker deployment device 640 during assembly.

While the introducer system 748 is illustrated without a hemostatic valve, it would be understood that the introducer system 748 may have a hemostatic valve, such as the valve 680. Collectively, the introducer 648, and spacers 630, 632 comprise an axial distance adjustment system that will permit a single length introducer, such as the introducer 648, to be used with medical devices having differing cannula lengths.

Accordingly, the depth of insertion of a multiple markers or the depth of performing multiple biopsies may be changed by removing or adding spacers 630 as illustrated in FIGS. 10 and 11, as the spacers 630 are used with either a biopsy device or a marker deployment device. This change of depth may be made without compromising the sterility of portions of the devices (such as the trocar tip 546) since the device inner cannula need not be removed from the spacer. Further, a tip, such as the trocar tip 546 will not be exposed to an event that may potentially dull the tip, since the device is not removed from the spacer to achieve different depths of insertion.

The present invention has been particularly shown and described with reference to the foregoing embodiments, which are merely illustrative of the best modes for carrying out the invention. It should be understood by those skilled in the art that various alternatives to the embodiments of the invention described herein may be employed in practicing the invention without departing from the spirit and scope of the invention as defined in the following claims. It is intended that the following claims define the scope of the invention and that the method and apparatus within the scope of these claims and their equivalents be covered thereby. This description of the invention should be understood to include all novel and non-obvious combinations of elements described herein, and claims may be presented in this or a later application to any novel and non-obvious combination of these elements. Moreover, the foregoing embodiments are illustrative, and no single feature or element is essential to all possible combinations that may be claimed in this or a later application.

1. A medical system, comprising:
A medical system, comprising: a first medical device having a first engaging portion and a first cannula extending therefrom;
a second medical device having a second engaging portion and a second cannula extending therefrom;
an introducer having an introducer cannula generally defined by an axis, a latch portion and a hub, wherein the introducer cannula is defined, at least in part, by an inner lumen, the hub includes a proximal end and a distal end, the introducer cannula includes an introducer cannula distal end and an introducer cannula proximal end, and wherein the introducer cannula proximal end is adjacent the hub; and
an axial distance adjustment system for providing an adjustable length between the introducer cannula distal end and at least a portion of one of the first engaging portion and the second engaging portion, wherein the adjustment system includes at least one latching spacer having a side opening for permitting the at least one spacer to be interposed between the introducer and at least one of-the first medical device and the second medical device, and wherein the latch portion is adapted to releasably secure the introducer to at least one of the latching spacer, the first medical device and the second medical device.

2. The system of claim 1, wherein at least one of the first cannula, of the first medical device, and a second cannula, of the second medical device, may be selectively interposed within the side opening when at least a portion of the at least one of the first cannula and the second cannula is at least partially interposed within the introducer cannula.

3. The system of claim 1, wherein at least a portion of the latch portion extends from the proximal end of the introducer.

4. The system of claim 1, wherein the first medical device includes a biopsy needle.

5. The system of claim 1, wherein the spacer includes a proximal spacer end and a distal spacer end and wherein the distal spacer end is selectively coupled with the introducer hub and the proximal end is selectively coupled with at least one of the first medical device and the second medical device.

6. The system of claim 5, wherein the side opening extends from the proximal spacer end to the distal spacer end.

7. The system of claim 1, wherein a portion of at least one of the first medical device and the second medical device is selectively coupled to the spacer such that at least a portion of the introducer cannula is axially restrained relative to at least a portion of at least one of the first medical device and the second medical device.

8. The system of claim 1, further comprising a second spacer, wherein the spacer includes a proximal spacer end and a distal spacer end, the second spacer includes a proximal second spacer end and a distal second spacer end, the distal spacer end is selectively coupled with the introducer hub, and wherein the proximal second end is selectively coupled with at least one of the first medical device and the second medical device.

9. The system of claim 1, wherein the latch portion will selectively engage the at least one of the first medical device and the second medical device with the introducer so as to axially restrain at least a portion of the at least one of the first medical device and the second medical device relative to the introducer cannula during at least a portion of a medical procedure.

10. The system of claim 1, wherein the system is constructed, at least in part, of a material that is magnetic resonance imaging (MRI) compatible.

11. A medical system comprising:
a first medical device having a first engaging portion and a first cannula extending therefrom;
a second medical device having a second engaging portion and a second cannula extending therefrom;
an introducer having a latch portion and an introducer cannula; and
an axial distance adjustment system for providing an adjustable length between the introducer cannula distal end and at least a portion of one of the first engaging portion and the second engaging portion, wherein the adjustment system includes at least one latching spacer having a side opening for permitting the at least one latching spacer to be interposed and releasably secured between the introducer and at least one of the first medical device and the second medical device, and wherein at least one of the first cannula and the second cannula may be selectively interposed within the side opening when at least a portion of the at least one of the first cannula and the second cannula is at least partially interposed within the introducer cannula.

12. The system of claim 11, wherein the introducer includes the latch portion and a hub, wherein the cannula is defined, at least in part, by an inner lumen, the latch portion is adapted to releasably secure the introducer to at least one of the first medical device and the second medical device, the hub includes a proximal end and a distal end, the introducer cannula includes an introducer cannula distal end and an introducer cannula proximal end, and wherein the introducer cannula proximal end is adjacent the hub.

13. The system of claim 11, further comprising the latch portion coupled to at least one of the introducer, the first medical device, and the second medical device, wherein the latch portion will selectively engage at least one of the first medical device and the second medical device with the introducer so as to axially restrain at least a portion of the at least one of the first medical device and the second medical device relative to the introducer cannula during at least a portion of a medical procedure.

14. The system of claim 13, wherein the latch portion will engage the first medical device with the introducer as the first medical device cannula is interposed within the introducer cannula, and wherein the latch may be manipulated so as to disengage the first medical device from the introducer so as to permit the first medical device cannula to be withdrawn from the introducer cannula.

15. A method of performing a medical procedure, comprising:
interposing a first medical device cannula into an introducer cannula
removing the first medical device from the introducer cannula;
interposing a second medical device cannula into the introducer cannula; and
interposing at least a portion of one of the first medical device cannula and the second medical device cannula into a portion of a latching spacer when the at least one of the first medical device cannula and the second medical device cannula is at least partially interposed within and latched to the introducer cannula.

16. The method of claim 15, further comprising latching a first medical device to the latching spacer with a latch such that axial movement is restricted between at least a portion of the first medical device and at least a portion of the introducer cannula, wherein the latch includes a release portion and manipulation of the release portion will permit unlatching of the introducer cannula from the first medical device.

17. The method of claim 15, further comprising inserting the introducer cannula into a patient's body in a pathway to a target tissue, and uncoupling the introducer cannula from a first medical device.

18. The method of claim 15, further comprising axially restraining at least a portion of the introducer cannula relative to at least one of the first medical device cannula and the second medical device.

19. The method of claim 15, further comprising removing a biopsy portion from a patient's body.

20. The method of claim 15, further comprising deploying the site marker within a biopsy site.
